(19) United States
(12) Patent Application Publication
(10) Pub. No.: US 2017/0014383 A1
(43) Pub. Date: Jan. 19, 2017

(54) SOLID DISPERSION OF INDIRUBIN

(71) Applicant: Phosphorex, Inc., Hopkinton, MA (US)

(72) Inventor: Bin Wu, Sharon, MA (US)

(73) Assignee: Phosphorex, Inc., Hopkinton, MA (US)

(21) Appl. No.: 15/186,631

(22) Filed: Jun. 20, 2016

Related U.S. Application Data
(63) Continuation of application No. PCT/US2014/071409, filed on Dec. 19, 2014.
(60) Provisional application No. 61/964,004, filed on Dec. 20, 2013.

(51) Int. Cl. A61K 31/404; A61K 9/16

(52) U.S. Cl. CPC A61K 31/404; A61K 9/1641

(57) ABSTRACT

The invention described herein provides a solid dispersion of Indirubin, a pharmaceutical composition comprising the solid dispersion of Indirubin, a process for preparing the solid dispersion and pharmaceutical composition, and a method of using the pharmaceutical composition.

### REFERENCE TO RELATED APPLICATION

This application is a continuation application of International Patent Application No. PCT/US2014/071409, filed on Dec. 19, 2014; which claims the benefit of the filing date of U.S. Provisional Applications No. 61/964,004, filed on Dec. 20, 2013, the entire content of each of which is incorporated herein by reference.

### BACKGROUND OF THE INVENTION

Indirubin is an extract from the indigo plant. Indirubin is a constituent of a traditional Chinese herbal formula, Dang Gui Long Hui Wan used in the treatment of chronic myelogenous leukemia (CML). It has also been used in Asia as a systemic treatment for psoriasis.

In vitro and animal studies of Indirubin have indicated anti-inflammatory, antitumor and neuroprotective effects. Researchers discovered that indirubin both blocks the migration of glioblastoma cells, preventing their spread to other areas of the brain, and the migration of endothelial cells, preventing them from forming the new blood vessels that the tumor needs to grow. Glioblastomas occur in about 18,500 Americans annually and kill nearly 13,000 of them. Glioblastoma multiforme is the most common and lethal form of the malignancy, with an average survival of 15 months after diagnosis.

Indirubin also inhibits cyclin-dependent kinases in tumor cells. A derivative of indirubin was shown to enhance the cytotoxic effects of adriamycin. A small clinical study of indirubin in patients with head and neck cancer found a reduction in mucosal damage from radiation therapy. Meisoindigo, a metabolite of indirubin, has also been shown to have similar properties. Positive effects following long term use of indirubin for the treatment of CML have been reported.

However, indirubin has a poor aqueous solubility and poor permeability, which limit its bioavailability, efficacy and delivery. Therefore, there exists a need in the art for indirubin formulations that can increase solubility, bioavailability, improve clinical efficacy, reduce patient dose variation, and potentially reduce side effects.

### SUMMARY OF THE INVENTION

A solid dispersion of Indirubin is provided herein. Also, a pharmaceutical composition comprising a solid dispersion of Indirubin is provided herein.

Further, a pharmaceutical composition comprising a solid dispersion of Indirubin is described herein. More specifically, a pharmaceutical composition comprising a solid dispersion of spray dried Indirubin is described herein.

Processes for preparing the solid dispersion and pharmaceutical composition and methods of using the pharmaceutical composition are also described herein.

It is contemplated that any embodiments described herein, including those only described under one aspect or those only in the examples, can be combined with any other embodiment(s) unless explicitly disclaimed.

### DETAILED DESCRIPTION OF THE INVENTION

Reference will now be made in detail below to describe certain aspects and exemplary embodiments of the invention. While enumerated embodiments will be described, it will be understood that they are not intended to limit the invention to those embodiments. On the contrary, the invention is intended to cover all alternatives, modifications, and equivalents. One skilled in the art will recognize many methods and materials similar or equivalent to those described herein, which could be used in the practice of the present invention. The present invention is in no way limited to the methods and materials described. In the event that one or more of the incorporated literature and similar materials differs from or contradicts this application, including but not limited to defined terms, term usage, described techniques, or the like, this application controls.

### DEFINITIONS

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of ˜5, 10, and up to 20%.

The term “amorphous” means a solid in a solid state that is a non-crystalline state. Amorphous solids generally possess crystal-like short range molecular arrangement, but no long range order of molecular packing as found in crystalline solids. The solid state form of a solid may be determined by polarized light microscopy, x-ray powder diffraction (“XRPD”), differential scanning calorimetry (“DSC”), or other standard techniques known to those of skill in the art.

The terms “cancer” and “cancerous” refer to or describe the physiological condition in mammals that is typically characterized by abnormal or unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, brain, endometrial or uterine carcinoma, salivary gland carcinoma, kidney or renal cancer, prostate cancer, vulval cancer, thyroid cancer, hepatic carcinoma, anal carcinoma, penile carcinoma, skin cancer, including melanoma, as well as head and neck cancer.

The phrase “dispersion polymer” means a polymer that allows for Indirubin to be dispersed throughout such that a solid dispersion may form. The dispersion polymer may contain a mixture of two or more polymers. Examples of dispersion polymers include, but are not limited to, vinyl polymers and copolymers, vinylpyrrolidine vinylacetate copolymer (“PVP-VA”), polyvinyl alcohols, polyvinyl alcohol polyvinyl acetate copolymers, polyvinyl pyrrolidine (“PVP”), acrylate and methacrylate copolymers, methylacrylic acid methyl methacrylate copolymer (such as Eudragit®), polyethylene polyvinyl alcohol copolymers, polyethylene glycol, polyoxyethylene-polyoxypropylene block copolymers (also referred to as poloxamers), graft copolymer comprised of polyethylene glycol, polyvinyl caprolactam and polyvinyl acetate (such as Soluplus®), cellulosic polymers, such as hydroxypropyl methyl cellulose acetate (“HPMCA”), hydroxypropyl methyl cellulose (“HPMC”), hydroxypropyl cellulose (“HPC”), methyl cellulose, hydroxyethyl methyl cellulose, hydroxyethyl cellulose, hydroxyethyl cellulose acetate, and hydroxyethyl ethyl cellulose, hypromellose acetate succinate (“HPMCAS”), hydroxypropyl methyl cellulose phthalate (“HPMCP”), carboxymethylethyl cellulose (“CMEC”), cellulose acetate phthalate (“CAP”), cellulose acetate succinate (“CAS”), hydroxypropyl methyl cellulose acetate phthalate (“HPMCAP”), cellulose acetate trimellitate (“CAT”), hydroxypropyl methyl cellulose acetate trimellitate (“HPMCAT”), and carboxymethylcellulose acetate butyrate (“CMCAB”), and the like. Said dispersion polymers may also be carbohydrates such as glucose, manitol, sucrose, etc.

The term “mammal” means a warm-blooded animal. The mammal may have or is at risk of developing a disease described herein. The mammal includes, but is not limited to, guinea pigs, dogs, cats, rats, mice, hamsters, and primates and other non-human mammals. Mammal may also include humans.

The phrase “pharmaceutically acceptable” indicates that the substance or composition is compatible chemically and/or toxicologically, with the other ingredients comprising a composition, and/or the mammal being treated therewith.

The phrase “pharmaceutically acceptable salt,” as used herein, refers to pharmaceutically acceptable organic or inorganic salts of a compound, such as those described herein.

The phrase “solid dispersion” means a system in a solid state comprising at least two components, wherein one component is dispersed throughout the other component. The solid dispersion discussed herein may comprise one component of Indirubin dispersed throughout another component, particularly a dispersion polymer.

The phrase “spray drying” refers to processes involved in breaking up liquid mixtures into small droplets (atomization) and rapidly removing solvent from the mixture in a spray drying apparatus where there is a strong driving force for evaporation of solvent from the droplets. The phrase spray drying is used conventionally and broadly. Spray drying processes and spray drying equipment are described generally in Perry, Robert H., and Don W. Green (eds.), Perry's Chemical Engineers' Handbook, New York: McGraw-Hill, 2007 (8^{th} edition).

The phrases “therapeutically effective amount” or “effective amount” mean an amount of a compound described herein that, when administered to a mammal in need of such treatment, sufficient to (i) treat or prevent the particular disease, condition, or disorder, (ii) attenuate, ameliorate, or eliminate one or more symptoms of the particular disease, condition, or disorder, or (iii) prevent or delay the onset of one or more symptoms of the particular disease, condition, or disorder described herein. The amount of a compound that will correspond to such an amount will vary depending upon factors such as the particular compound, disease condition and its severity, the identity (e.g., weight) of the mammal in need of treatment, but can nevertheless be routinely determined by one skilled in the art.

The terms “treat” or “treatment” refer to therapeutic, prophylactic, palliative or preventative measures, preferably therapeutic measures. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder, as well as those prone to have the condition or disorder or those in which the condition or disorder is to be prevented.

### Solid Dispersions and Pharmaceutical Compositions

Provided herein is a solid dispersion comprising Indirubin.

The solid dispersions are generally prepared by dissolving the drug substance and the dispersion polymer in a suitable solvent to form a feed solution, and then the feed solution may be spray dried to form the solid dispersion (and remove the solvent). Spray drying is a known process. Spray drying is generally performed by dissolving Indirubin and the dispersion polymer in a suitable solvent to prepare a feed solution. The feed solution may be pumped through an atomizer into a drying chamber. The feed solution can be atomized by conventional means known in the art, such as a two-fluid sonicating nozzle, a pressure nozzle, a rotating nozzle and a two-fluid non-sonicating nozzle. Then, the solvent is removed in the drying chamber to form the solid dispersion. A typical drying chamber uses hot gases, such as forced air, nitrogen, nitrogen-enriched air, or argon to dry particles. The size of the drying chamber may be adjusted to achieve particle properties or throughput.

Although the solid dispersion are preferably prepared by conventional spray drying techniques, other techniques known in the art may be used, such as melt extrusion, freeze drying, rotary evaporation, drum drying or other solvent removal processes.

In one embodiment, a process of preparing a solid dispersion is provided, comprising:

(a) dissolving Indirubin and a dispersion polymer in a solvent to form a feed solution; and

(b) removing the solvent to form the solid dispersion.

In a further embodiment, the removal of the solvent in step (b) is performed by spray drying, melt extrusion, freeze drying, rotary evaporation, drum drying or other solvent removal processes.

In another embodiment, the removal of the solvent is achieved by a precipitation method (“precipitation”). For example, the polymer-Indirubin solution can be mixed with another liquid in which both the polymer and Indirubin have low solubility. The polymer-Indirubin solid dispersion forms as they precipitate in said liquid. Such formed solid dispersion can be purified and collected by, for example, centrifugation, decanting, drying and/or lyophilization. See the HCl used in Examples 1 and 2.

In certain embodiments, the dispersion polymer is selected from PVP, PVP-VA, methylacrylic acid methyl methacrylate copolymer, anionic copolymers of methacrylic acid and methyl methacrylate (e.g., EUDRAGIT® L100), HPMCP, CAP, HPMCAS, HPMC, manitol and mixtures thereof.

Suitable solvents are a solvent or mixture of solvents in which both Indirubin and the dispersion polymer have adequate solubility (solubility greater than 1 mg/mL). A mixture of solvents may be used if each component of the solid dispersion (i.e. Indirubin and dispersion polymer) requires different solvents to obtain the desired solubility. The solvent may be volatile with a boiling point of 150° C. or less. In addition, the solvent should have relatively low toxicity and be removed from the dispersion to a level that is acceptable to The International Committee on Harmonization (“ICH”) guidelines. Removal of solvent to this level may require a subsequent processing step, such as tray drying.

Examples of suitable solvents include, but are not limited to, alcohols, such as methanol (“MeOH”), ethanol (“EtOH”), n-propanol, isopropanol (“IPA”) and butanol; ketones, such as acetone, methyl ethyl ketone (“MEK”) and methyl isobutyl ketone; esters, such as ethyl acetate (“EA”) and propyl acetate; and various other solvents, such as tetrahydrofuran (“THF”), acetonitrile (“ACN”), methylene chloride, toluene and 1, 1, 1-trichloroethane. Lower volatility solvents, such as dimethyl acetate or dimethylsulfoxide (“DMSO”), N,N-Dimethylacetamide (“DMA”), or dimethylformamide (“DMF”), may be used. Mixtures of solvents with water may also be used, so long as the polymer and Indirubin are sufficiently soluble to make the spray drying process practicable. Generally, due to the hydrophobic nature of low solubility drugs, non-aqueous solvents may be used, meaning the solvent comprises less than about 10 weight % water.

In certain embodiments, the amount of Indirubin in the solid dispersion ranges from about 0.1% to about 95% by weight relative to the dispersion polymer.

In certain embodiments, the amount of Indirubin in the solid dispersion ranges from 1% to 60% by weight relative to the dispersion polymer.

In certain embodiments, the amount of Indirubin in the solid dispersion ranges from about 5% to about 50% by weight relative to the dispersion polymer.

In certain embodiments, the solid dispersion is an amorphous solid dispersion.

Another embodiment provides a pharmaceutical composition comprising a solid dispersion of Indirubin and a dispersion polymer, and a pharmaceutically acceptable carrier or excipient.

Suitable carriers and excipients are well known to those skilled in the art and are described in detail in, e.g., Ansel, Howard C. et al., Ansel's Pharmaceutical Dosage Forms and Drug Delivery Systems, Philadelphia: Lippincott, Williams & Wilkins, 2004; Gennaro, Alfonso R. et al., Remington: The Science and Practice of Pharmacy, Philadelphia: Lippincott, Williams & Wilkins, 2000; and Rowe, Raymond C., Handbook of Pharmaceutical Excipients, Chicago, Pharmaceutical Press, 2005.

The pharmaceutical compositions may also include one or more additional components, such as buffers, dispersion agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug, i.e., a compound described herein or pharmaceutical composition thereof, or aid in the manufacturing of the pharmaceutical product, i.e., medicament (see Ansel; Gennaro; and Rowe above). The components of the pharmaceutical composition should be pharmaceutically acceptable.

Certain embodiments provide a pharmaceutical composition comprising:

(a) about 1 to about 70 weight % of a solid dispersion of Indirubin;

(b) about 0.1 to about 20 weight % of a disintegrant;

(c) about 0.1 to about 25 weight % of an osmogen;

(d) about 0.1 to about 10 weight % of a glidant;

(e) about 0.1 to about 10 weight % of a lubricant; and

(f) about 0.1 to about 25 weight % of a binder/diluent.

The pharmaceutical composition preferably contains a therapeutically effective amount of Indirubin. However, in some embodiments, each individual dose contains a portion of a therapeutically effective amount of Indirubin, such that multiple doses of the composition may be required (for example, two or more tablets are required for a therapeutically effective amount). Thus, in this application, when it states that the pharmaceutical composition contains a therapeutically effective amount, it may mean that the composition may be one dose (for example, one tablet) or multiple doses (for example, two tablets).

In certain embodiments, the pharmaceutical composition contains about 0.1-500 mg of Indirubin, about 1-100 mg of Indirubin, about 2-50 mg of Indirubin, or about 5-25 mg of Indirubin.

The pharmaceutical compositions described herein may be administered by any convenient route appropriate to the condition to be treated. Suitable routes include oral, parenteral (including subcutaneous, intramuscular, intravenous, intraarterial, intradermal, intrathecal and epidural), transdermal, rectal, nasal, topical (including buccal and sublingual), ocular, vaginal, intraperitoneal, intrapulmonary and intranasal. If parenteral administration is desired, the compositions will be sterile and in a solution or suspension form suitable for injection or infusion.

The compounds may be administered in any convenient administrative form, e.g., tablets, powders, capsules, dispersions, suspensions, syrups, sprays, suppositories, gels, emulsions, patches, etc.

The pharmaceutical compositions described herein are typically administered orally. The pharmaceutical compositions described herein are typically administered as a tablet, caplet, hard or soft gelatin capsule, pill, granules or a suspension.

### Methods of Treatment

Also provided are methods of treating or preventing disease or condition by administering the pharmaceutical composition described herein. In one embodiment, a human patient is treated with a pharmaceutical composition described herein for cancer.

In another embodiment, a method of treating or preventing cancer in a mammal in need of such treatment, wherein the method comprises administering to said mammal a pharmaceutical composition described herein. The cancer is breast cancer, ovarian cancer, cervical cancer, prostate cancer, testicular cancer, genitourinary tract cancer, esophagus cancer, larynx cancer, glioblastoma, neuroblastoma, stomach cancer, skin cancer, keratoacanthoma, lung cancer, epidermoid carcinoma, large cell carcinoma, NSCLC, small cell carcinoma, lung adenocarcinoma, bone cancer, colon cancer, adenoma, pancreatic cancer, adenocarcinoma, thyroid cancer, follicular carcinoma, undifferentiated carcinoma, papillary carcinoma, seminoma, melanoma, sarcoma, bladder carcinoma, liver carcinoma, biliary passage cancer, kidney carcinoma, myeloid cancer, lymphoid cancer, chronic myelogenous leukemia (CML), hairy cell cancer, small intestine cancer, colorectal cancer, large intestine cancer, rectal cancer, brain cancer, head and neck cancer, central nervous system cancer, Hodgkin's lymphoma, leukemia, or a cancer of the buccal cavity, pharynx (oral), lip, tongue, mouth, or pharynx.

Another embodiment provides the use of a pharmaceutical composition described herein, in the manufacture of a medicament for the treatment of cancer.

In another embodiment, a method of treating or preventing neurodegenerative disorders in a mammal in need of such treatment, wherein the method comprises administering to said mammal a pharmaceutical composition described herein. More specifically, said neurodegenerative disorder is selected from Alzheimer's disease and Parkinson's disease.

In yet another embodiment, the method of the invention can be used to treat an inflammatory disease or autoimmune disease, such as psoriasis or psoriatic arthritis.

### EXAMPLES

The examples described herein are for illustration only and are not limiting.

### Example 1

Approximately 2.5 mg of Indirubin was dissolved in 1 mL of dimethylacetamide (DMA). Such solution was sonicated for 5 minutes to ensure complete dissolution. Approximately 18 mg of hypromellose acetate succinate (HPMCAS) was dissolved in the same 1 mL DMA solution, and was poured into a 50 mL container containing 10 mL of 0.01 N HCl. Upon mixing, a precipitate formed immediately. The mixture was centrifuged for 3 minutes at 1000×g. The supernatant was decanted, and 35 mL of 0.01 N HCl was added tore-suspend the pellet. The resulting suspension was centrifuged again for 3 minutes at 1000×g, and the supernatant was decanted and another 35 mL of 0.01 N HCl was added to re-suspend the pellet. Finally, the mixture was centrifuged for 3 minutes at 1300×g, the supernatant was decanted, and the resulting pellet was freeze-dried. A total of 5.7 mg of the final solid dispersion was obtained.

### Example 2

20 mL of DMA was added to a vial containing approximately 77.2 mg of Indirubin. The resulting solution was sonicated for 5 minutes to ensure complete dissolution. 1 g of HPMCAS was added to form a clear solution of Indirubin and HPMCAS. The Indirubin/HPMCAS solution was added to 200 mL of 0.1 M HCl solution while stirring. The mixture was filtered using 0.45μ filter paper, rinsed with 0.1 M HCl, and transferred to two 50 mL centrifuge tubes. Tubes were spun at 500 rpm for 5 minutes. The supernatant in the tubes was decanted, and then 45 mL of 0.1 M HCl was added to each tube to re-suspend the pellets. The tubes were centrifuged again at 2,000 rpm for 5 minutes. After the supernatant was decanted, the pellets in both tubes were freeze-dried. Approximately 850 mg of dry product of solid dispersion was obtained.

1. A nanoparticulate indirubin composition comprising:
(a) particles of indirubin or derivatives thereof, wherein the indirubin particles have an effective average particle size of less than 2 microns; and
(b) at least one surface stabilizer.

2. The composition of claim 1, wherein the effective average particle size of the nanoparticulate indirubin particles is less than 1000 nm, or less than 500 nm.

3. The composition of claim 1, wherein the effective average particle size of the nanoparticulate indirubin particles is less than 500 nm.

4. The composition of claim 1, wherein at least about 70% of the indirubin particles have a particle size less than the effective average particle size.

5. The composition of claim 1, wherein the composition is formulated for oral or intravenous administration.

6. The composition of claim 1, wherein the composition further comprises one or more pharmaceutically acceptable excipients, carriers, or a combination thereof.

7. The composition of claim 1, wherein the indirubin is present from about 80% to about 99.9999% by weight, based on the total combined dry weight of the indirubin and at least one surface stabilizer, not including other excipients.

8. The composition of claim 1, wherein the at least one surface stabilizer is present in an amount of from about 0.0001% to about 20% by weight, based on the total combined dry weight of the indirubin and at least one surface stabilizer, not including other excipients.

9. The composition of claim 1, comprising at least two surface stabilizers.

10. The composition of claim 1, wherein the surface stabilizer is selected from the group consisting of an anionic surface stabilizer, a cationic surface stabilizer, a zwitterionic surface stabilizer, and a non-ionic surface stabilizer.

11. The composition of claim 1, wherein the at least one surface stabilizer is a poloxamer.

12. The composition of claim 11, wherein the poloxamer is selected from the group consisting of block copolymers of ethylene oxide and propylene oxide.

13. The composition of claim 11, wherein the poloxamer is a block copolymer of ethylene oxide.

14. The composition of claim 1, further comprising an indirubin composition having an effective average particle size of greater than 2 microns.

15. The composition of claim 1, further comprising at least one additional nanoparticulate indirubin composition having an effective average particle size of less than 2 microns, wherein said additional nanoparticulate indirubin composition has an effective average particle size which is different than the effective average particle size of the nanoparticulate indirubin composition of claim 1.

16-19. (canceled)

20. A method of making the nanoparticulate indirubin composition of claim 1, comprising contacting indirubin particles with at least one surface stabilizer for a time and under conditions sufficient to provide a nanoparticulate indirubin composition having an effective average particle size of less than 2 microns.

21-24. (canceled)

25. A method of treating cancer, an inflammatory disease, or a neurodegenerative disorder in a subject in need thereof comprising administering an effective amount of the nanoparticulate indirubin composition of claim 1.

26-34. (canceled)
